(19) United States
(12) Patent Application Publication
(10) Pub. No.: US 2012/0197142 A1
(43) Pub. Date: Aug. 2, 2012

(54) Method And System For Determining Vascular Changes Using Plethysmographic Signals

(75) Inventors: David Lovejoy, Thiensville, WI (US); Shannon Campbell, Boulder, CO (US)

(73) Assignee: Nellcor Puritan Bennett LLC, Boulder, CO (US)

(21) Appl. No.: 13/017,462

(22) Filed: Jan. 31, 2011

(51) Int. Cl. A61B 5/02

(52) U.S. Cl. 600/507

(57) ABSTRACT

Embodiments of the present disclosure relate to determining changes in a vasculature by analyzing changes in one or more attributes of a plethysmographic signal. According to certain embodiments, an apparatus may obtain plethysmographic signals prior to and subsequent to the administration of a vasoactive stimulus. The apparatus may include a processing unit configured to analyze and to compare attributes of the plethysmographic signals. The processing unit may correlate any changes in signal attributes to a change in a physiological condition of the vasculature (e.g., vascular tone/compliance). The apparatus may also include a display unit configured to present any changes in the vasculature as well as any alarms in response to a trigger from the processing unit.

### BACKGROUND

The present disclosure relates generally to medical devices and, more particularly, to methods of analyzing one or more attributes of plethysmographic signals and correlating these attributes to a physiological condition.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption of the transmitted light in such tissue. A typical pulse oximeter may use light emitting diodes (LEDs) to measure light absorption by the blood. The absorbed and/or scattered light may be detected by the pulse oximeter, which may generate a signal that is proportional to the intensity of the detected light.

A typical signal resulting from the sensed light may be referred to as a plethysmographic waveform. Valuable clinical data may be obtained from the morphology of the plethysmographic waveform relating to specific physiological parameters of the patient. Accordingly, it may desirable to monitor changes in the morphology of the plethysmographic waveform to determine changes in specific physiological parameters of the patient.

### BRIEF DESCRIPTION OF THE DRAWINGS

Advantages of the disclosed techniques may become apparent upon reading the following detailed description and upon reference to the drawings in which:

FIG. 1 is a perspective view of a pulse oximetry system in accordance with an embodiment;

FIG. 2 is a simplified block diagram of an embodiment of the pulse oximetry system in FIG. 1 coupled to a patient;

FIG. 3 is a flow chart depicting an embodiment of a method for determining vascular tone/compliance in response to a vasoactive stimulus based on attributes of a plethysmographic signal;

FIG. 4A is a representation of the plethysmographic signal with multiple amplitudes;

FIG. 4B is a representation of a length in duration of the plethysmographic signal;

FIG. 4C is a representation of an area of the plethysmographic signal;

FIG. 4D is a representation of changes in sharpness of the plethysmographic signal;

FIG. 4E is a representation of changes in slope of the plethysmographic signal;

FIG. 4F is a representation of movement of a dicrotic notch along the plethysmographic signal;

FIG. 4G is a representation of oscillation or twisting of the plethysmographic signal;

FIG. 5 is a representation of an embodiment of a display providing a graphical indicator related to compliance;

FIG. 6 is a representation of an embodiment of a display providing a graphical indicator related to compliance;

FIG. 7 is a representation of an embodiment of a display providing a graphical indicator related to compliance;

FIG. 8 is a representation of an embodiment of a display providing a graphical indicator related to compliance;

FIG. 9 is a block diagram of a closed-loop system to manage compliance; and

FIG. 10 is a flow chart depicting an embodiment of a method for determining and managing compliance using the closed-loop system in FIG. 9.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to determining information from a patient's physiological signal based on a processing and/or comparison of signal features from the physiological signal. More specifically, a physiological signal is generated by a physiological monitoring system, such as pulse oximeter, in response to light that is detected after being emitted and transmitted through the patient's tissue. The physiological signal, typically a plethysmographic signal or waveform, may be processed using an algorithm and signal processing techniques to determine various physiological parameters. Normal processing of the plethysmographic signal may enable analyses of certain signal attributes such as amplitude or frequency. However, more advanced techniques may allow derivation of further attributes and information from the signal. Utilizing algorithms and advanced signal processing techniques, attributes of the original plethysmographic signal may be analyzed. For example, information may be produced regarding the area under the curve of the signal, changes in the slope of the upstroke and downstroke segments of the signal, or position of the dicrotic notch. Using these techniques, valuable clinical data may be derived from the plethysmographic signal relating to one or more specific physiological parameters, such as a change in arterial system compliance in response to a vasoactive stimulus.

The plethysmographic signal may be analyzed for changes in signal attributes by comparing the plethysmographic signal to a baseline signal. The baseline plethysmographic signal may be obtained prior to the administration of a vasoactive drug or local anesthetic agent. Signal processing techniques may be utilized to determine whether attributes of the plethysmographic signal differ substantially from the baseline plethysmographic signal, which may indicate a change in the physiological state of the patient. For example, differences in signal attributes may relate to changes in the compliance or vasculature tone of a patient in response to a vasoactive drug, to changes in the cardiovascular and central nervous toxicity following intravasculature injection of local anesthetic solutions, or to changes in blood pressure and oxygen saturation as it relates to the depth of anesthesia. As analysis of the plethysmographic signal may enable analyses of multiple signal attributes, analysis may also include comparisons of multiple signals received from multiple sites on the patient. Such multi-signal analyses may provide additional information related to physiological responses in different vasculatures, for example, the response in a central vasculature versus a peripheral vasculature. In certain embodiments, a higher resolution signal (e.g., a continuous wavelet transformed signal) that provides richer data content may also be used in analyzing the original plethysmographic signal.

Data processing circuitry may generate physiological data based on the attributes of the obtained plethysmographic signal as well as from comparison of this signal to the baseline signal, A monitor may also contain a display capable of showing the original plethysmographic signal or the high resolution signal without having to autoscale to display the signal. In some embodiments, the display may be configured to provide an indication of any change in the physiological data of a patient based upon the comparison of attributes of the plethysmographic signal to the baseline plethysmographic signal. Further, the monitor may provide an alarm due to changes in the physiological data.

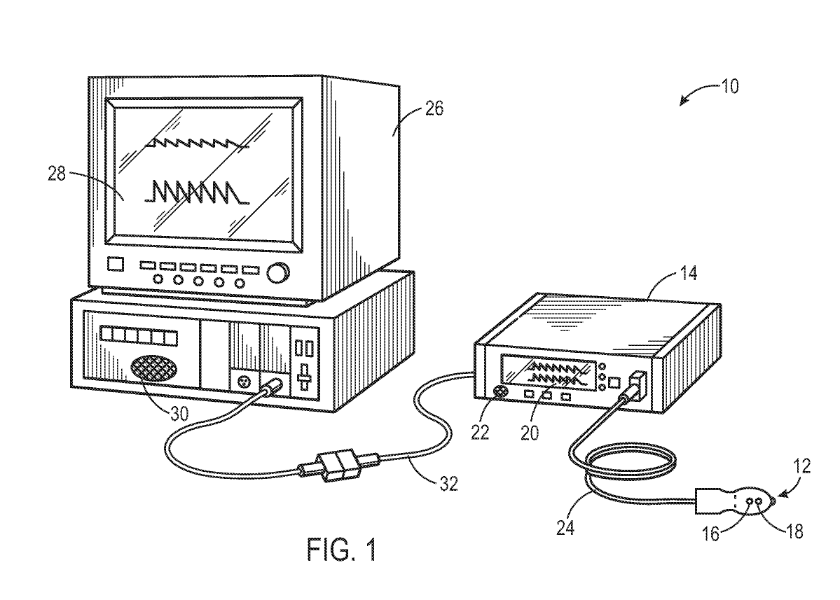

FIG. 1 is a perspective view of a pulse oximetry system 10 in accordance with an embodiment. The system 10 may include a sensor 12 and a pulse oximetry monitor 14. The sensor 12 may include an emitter 16 for emitting light at certain wavelengths into a patient's tissue and a detector 18 for detecting the light after it is reflected and/or absorbed by the patient's tissue. In certain embodiments, the system 10 may include multiple sensors 12 instead of the single sensor 12. The monitor 14 may be capable of calculating physiological characteristics received from the sensor 12 relating to light emission and detection. Further, the monitor 14 may include a display 20 capable of displaying the physiological characteristics, other information about the system, and/or alarm indications. The monitor 14 also may include a speaker 22 to provide an audible alarm in the event that the patient's physiological characteristics exceed a threshold. The sensor 12 may be communicatively coupled to the monitor 14 via a cable 24. However, in other embodiments a wireless transmission device or the like may be utilized instead of or in addition to the cable 24.

In the illustrated embodiment, the pulse oximetry system 10 also may include a multi-parameter patient monitor 26. In addition to the monitor 14, or alternatively, the multi-parameter patient monitor 26 may be capable of calculating physiological characteristics and providing a central display 28 for information from the monitor 14 and from other medical monitoring devices or systems. For example, the multi-parameter patient monitor 26 may display a patient's SpO₂ and pulse rate information from the monitor 14 and blood pressure from a blood pressure monitor on the display 28. Additionally, the multi-parameter patient monitor 26 may indicate an alarm condition via the display 28 and/or a speaker 30 if the patient's physiological characteristics are found to be outside of the normal range. The monitor 14 may be communicatively coupled to the multi-parameter patient monitor 26 via a cable 32 coupled to a sensor input port or a digital communications port. In addition, the monitor 14 and/or the multi-parameter patient monitor 26 may be connected to a network to enable the sharing of information with servers or other workstations.

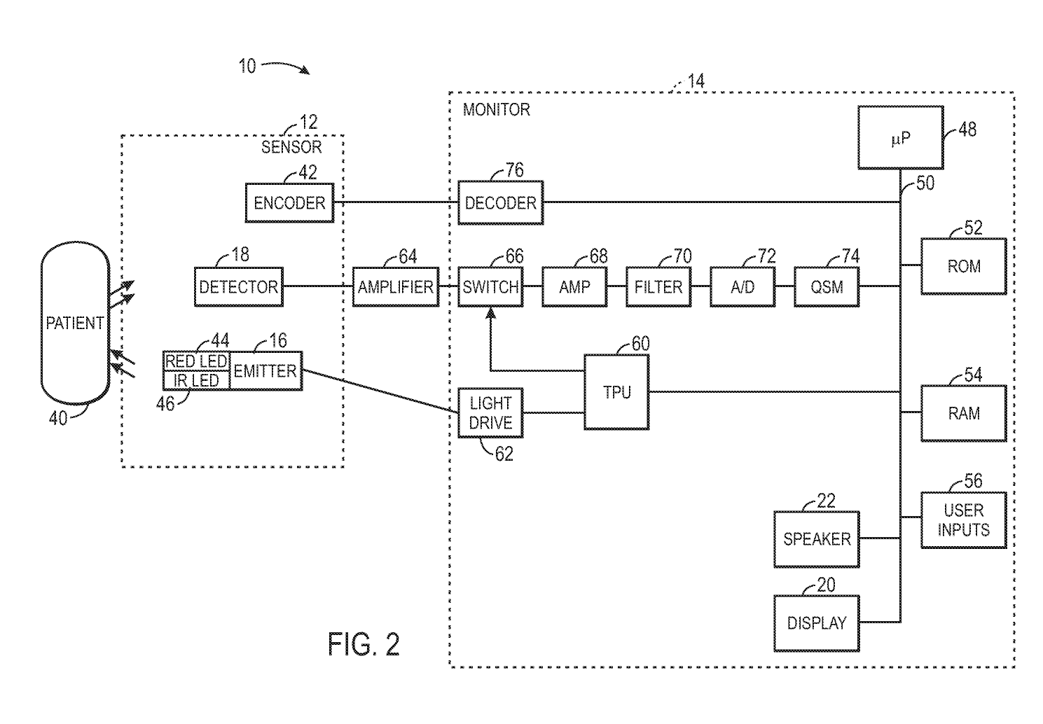

FIG. 2 is a block diagram of the pulse oximetry system 10 of FIG. 1 coupled to a patient 40 in accordance with present embodiments. Examples of pulse oximeters that may be used in the implementation of the present disclosure include pulse oximeters available from Nellcor Puritan Bennett LLC, but the following discussion may be applied to other pulse oximeters and medical devices. Specifically, certain components of the sensor 12 and the monitor 14 are illustrated in FIG. 2. The sensor 12 may include the emitter 16, the detector 18, and an encoder 42. It should be noted that the emitter 16 may be capable of emitting at least two wavelengths of light, e.g., RED and IR, into a patient's tissue 40. Hence, the emitter 16 may include a RED LED 44 and an IR LED 46 for emitting light into the patient's tissue 40 at the wavelengths used to calculate the patient's physiological characteristics. In certain embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 18 may be capable of detecting certain wavelengths of light. In another example, the detector 18 may detect a wide spectrum of wavelengths of light, and the monitor 14 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 18 may be capable of detecting the intensity of light at the RED and IR wavelengths. In operation, light enters the detector 18 after passing through the patient's tissue 40. The detector 18 may convert the intensity of the received light into an electrical signal. The light intensity may be directly related to the absorbance and/or reflectance of light in the tissue 40. That is, when more light at a certain wavelength is absorbed or reflected, less light of that wavelength is typically received from the tissue by the detector 18. After converting the received light to an electrical signal, the detector 18 may send the signal, which may be a plethysmographic (“pleth”) signal, to the monitor 14, where physiological characteristics may be calculated based at least in part on the absorption of the RED and IR wavelengths in the patient's tissue 40.

The encoder 42 may contain information about the sensor 12, such as what type of sensor it is (e.g., whether the sensor is intended for placement on a forehead or digit) and the wavelengths of light emitted by the emitter 16. This information may allow the monitor 14 to select appropriate algorithms and/or calibration coefficients for calculating the patient's physiological characteristics. The encoder 42 may, for instance, be a coded resistor which stores values corresponding to the type of the sensor 12 and/or the wavelengths of light emitted by the emitter 16. These coded values may be communicated to the monitor 14, which determines how to calculate the patient's physiological characteristics. In another embodiment, the encoder 42 may be a memory on which one or more of the following information may be stored for communication to the monitor 14: the type of the sensor 12; the wavelengths of light emitted by the emitter 16; the proper calibration coefficients and/or algorithms to be used for calculating the patient's physiological characteristics; baseline plethysmographic signal of the patient; patient history; historical trends; specific attributes of plethysmographic signals obtained from the patient; and algorithms for analyzing the morphology of the plethysmographic signal and correlating changes in this morphology to changes in the patient's physiological characteristics. The memory may be mapped with certain locations dedicated to information such as the type of sensor or the proper calibration coefficients. Other locations within the memory may be available for information such as the baseline plethysmographic signal and/or baseline signal attributes of the patient or historical trends (e.g., plethysmographic signals obtained from the patient). Pulse oximetry sensors capable of cooperating with pulse oximetry monitors include the OxiMax® sensors available from Nellcor Puritan Bennett LLC.

Signals from the detector 18 and the encoder 42 may be transmitted to the monitor 14. For example, the monitor 14 may access the mapped memory of the encoder 42 to obtain the baseline plethysmographic signal or specific baseline signal attributes of the patient and/or historical data relating to plethysmographic signals obtained from the patient. The monitor 14 generally may include one or more processors 48 connected to an internal bus 50. Also connected to the bus may be a read-only memory (ROM) 52, a random access memory (RAM) 54, user inputs 56, the display 20, or the speaker 22. A time processing unit (TPU) 60 may provide timing control signals to a light drive circuitry 62 which controls when the emitter 16 is illuminated and the multiplexed timing for the RED LED 44 and the IR LED 46. The TPU 60 controls the gating-in of signals from detector 18 through an amplifier 64 and a switching circuit 66. These signals may be sampled at the proper time, depending upon which light source is illuminated. The received signal from the detector 18 may be passed through an amplifier 68, a low pass filter 70, and an analog-to-digital converter 72. The digital data may then be stored in a queued serial module (QSM) 74 for later downloading to the RAM 54 as the QSM 74 fills up. In one embodiment, there may be multiple separate parallel paths having the amplifier 68, the filter 70, and the A/D converter 72 for multiple light wavelengths or spectra received.

The processor(s) 48 may determine the patient's physiological characteristics, such as SpO₂ and pulse rate, using various algorithms and/or look-up tables based generally on the value of the received signals corresponding to the light received by the detector 18. In certain embodiments, the processor(s) 48 may derive a desired physiological condition (e.g., arterial system compliance) based on one or more features (e.g., position of dicrotic notch) from received signals or a transformed versions (i.e., higher resolution) of the signals. For example, higher resolution signals may be obtained via continuous wavelet transformation as disclosed in U.S. application Ser. No. 12/437,317, titled “Concatenated Scalograms,” filed May 7, 2009, and incorporated herein by reference in its entirety for all purposes. In some embodiments, information may be derived from a selected portion (e.g., ascending limb) or portions of the received original signal (or higher resolution signal) and compared to a related portion of a subsequently received original (or higher resolution) signal following an event (e.g., administration of a vasoactive drug) to correlate the changes in the signal attributes to a change in a physiological condition (e.g., arterial system compliance). Embodiments of the present disclosure may utilize systems and methods such as those disclosed in U.S. application Ser. No. 12/437,317, for obtaining information from the received signal to determine and to detect changes in physiological conditions. For example, the processor(s) 48 use one or more algorithms for analyzing and measuring attributes of the plethysmographic signal as well as correlating changes in these attributes to a physiological condition, such as vascular tone/compliance. These algorithm(s) may be provided by the encoder memory to the processor(s) 48.

Signals corresponding to information about the sensor 12 may be transmitted from the encoder 42 to a decoder 76. The decoder 76 may translate these signals to enable the processor(s) to determine the proper method for calculating the patient's physiological characteristics, for example, based generally on algorithms or look-up tables stored in the ROM 52 (e.g., algorithms for correlating changes in the plethysmographic signal attributes to a physiological condition). In addition, or alternatively, the encoder 42 may contain the algorithms or look-up tables for calculating the patient's physiological characteristics. Further, the encoder 42 may provide the baseline plethysmographic signal and/or baseline signal attributes of the patient or historical data relating to plethysmographic signals from the patient.

As mentioned above, certain physiological conditions may be determined by analyzing attributes of the plethysmographic signal. For example, arterial system compliance may be determined. The autonomic nervous system is responsible for maintaining normal arterial pressure. The autonomic nervous system includes two components, the sympathetic system and the parasympathetic system. Both of these components monitor and control arterial blood pressure, heart rate, and respiration rate. Under normal conditions, the sympathetic system maintains a partial contraction of the blood vessels. However, in response to stress, the sympathetic system becomes a vasoconstrictor resulting in arterial constriction, thus increasing peripheral resistance and arterial pressure. The parasympathetic system regulates conservative processes and is usually active during relaxation or sleep. The parasympathetic system is generally responsible for decreasing heart rate, cardiac output, and respiration. The amplitude and morphology of the plethysmographic signal may correlate to changes in blood volume and vascular compliance. The autonomic nervous system modulates these changes and thus the attributes of the plethysmographic signal.

The pulse oximetry system 10 illustrated in FIG. 2 may be employed to measure and analyze attributes of the plethysmographic signal to determine vascular tone or compliance. Compliance and vascular tone are interrelated. Compliance is measured as an increase in volume over a change in pressure (e.g., mL/mm Hg). Vascular tone is the amount a blood vessel constricts relative to its maximal dilation. All arterial and venous vessels under normal conditions exhibit some amount of smooth muscle contraction that determines the diameter and, thus, the tone of the vessel. Compliance of vessels decreases at higher pressures and volumes. In addition, compliance of veins and arteries are similar at higher pressures and volumes. At lower pressures and volumes, compliance of veins is significantly greater than arteries. Increases in vascular tone result in decreases in compliance. In the arteries, a decrease in compliance (increase in vascular tone) decreases arterial blood volume and increases arterial blood pressure.

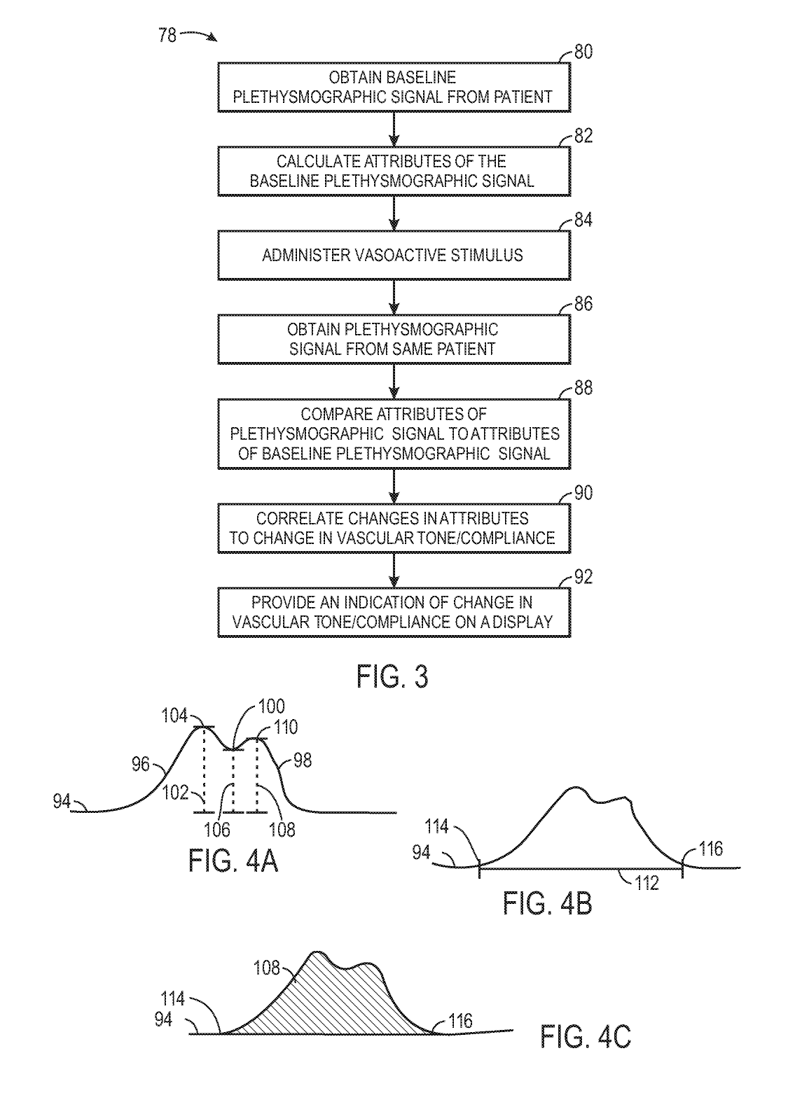

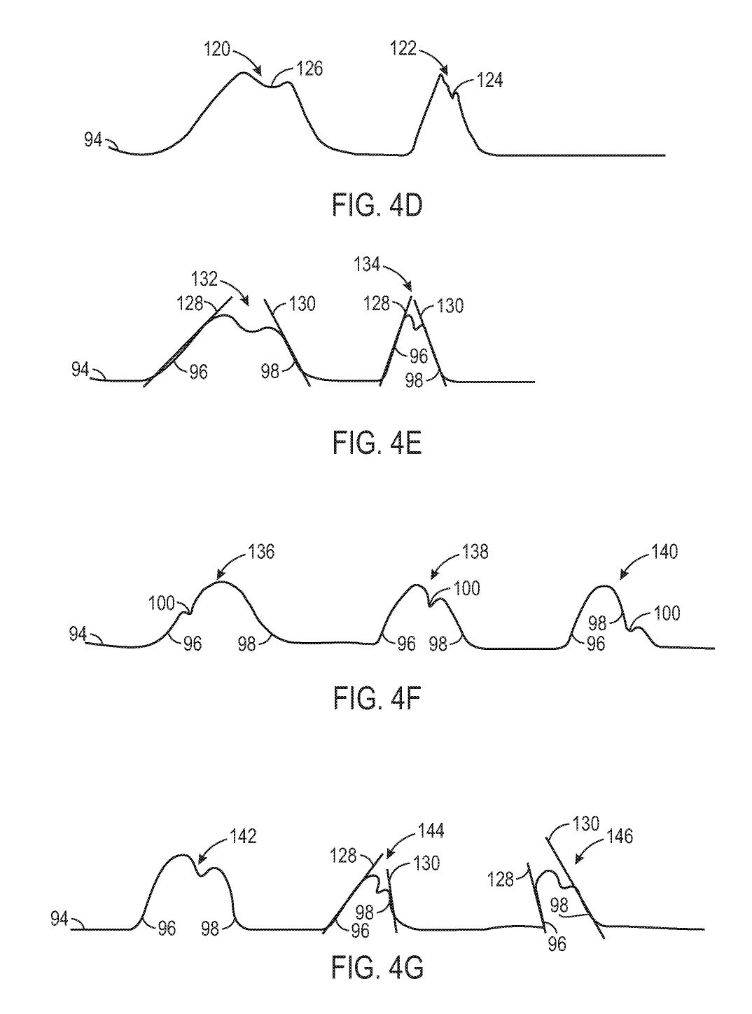

Vascular tone of an artery may be the product of both extrinsic factors (i.e., originating from outside the organ or tissue) and intrinsic factors (i.e., originating from the surrounding organ or tissue). In particular, the state of vascular tone and, thus, compliance, is determined by factors that influence constriction and dilation (e.g., a vasoactive stimulus or drug). FIG. 3 depicts an embodiment of a method 78 for determining the vascular tone/compliance in response to a vasoactive stimulus or drug from the attributes of the plethysmographic signal. In general, the method 78 may begin by obtaining a baseline plethysmographic signal from a patient 40 (block 80). The baseline plethysmographic signal may be the original signal or a higher resolution signal. Upon obtaining the baseline signal, attributes of the baseline plethysmographic signal may be calculated (block 82). For example, as shown in FIG. 4A, changes in amplitude of systolic and diastolic peaks 104 and 110, respectively, of the plethysmographic signal may reflect changes in compliance. For example, distances from the peaks 104 and 110 to the trough increase as the vasculature becomes more complaint and blood volume increases. In certain embodiments, the portions of the signals corresponding to peaks 104 and 110 may be selected and further processed as described in U.S. application Ser. No. 12/437,317. FIGS. 4B-G illustrate further examples of signal attributes that may be measured to determine compliance. A vasoactive stimulus or drug may be administered to the patient (block 84) after calculating the attributes of the baseline signal. The vasoactive stimulus may include a vasodilator (e.g., nitroglycerin) or vasoconstrictor (e.g., norepinephrine). Also, the vasoactive stimulus may include an anesthetic solution.

Following application of the vasoactive stimulus, a plethysmographic signal may be obtained from the same patient (block 86). As with the baseline plethysmographic signal, the plethysmographic signal may be the original signal or a higher resolution signal. Also, similarly, the same attributes may be calculated from the plethysmographic signal as Were calculated from the baseline plethysmographic signal. Following processing, the calculated attributes from the plethysmographic signal may then be compared to calculated attributes of the baseline signal (block 88). Upon comparing the attributes from both signals, changes in these attributes may be correlated to a change in vascular tone/compliance (block 90).

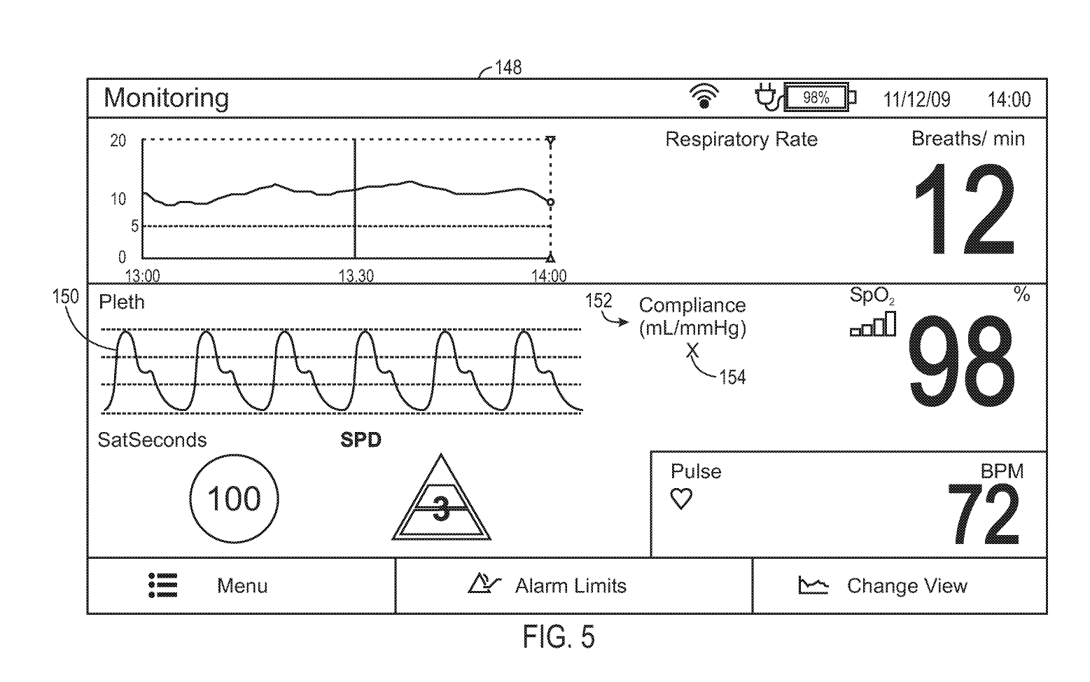

A measurement of compliance as well as any change in compliance may be provided on the display 20 of the monitor 14 (block 92), as described below in FIGS. 5-8. The monitor 14 may also provide an alarm. The alarm may include an audible alarm via speakers 22 or a visual alarm via display 20.

FIGS. 4A-G depict various attributes of the plethysmographic signal that may be analyzed as described above. One or more of the attributes below as well as other attributes not mentioned may be used in the described embodiments. As described below, changes in these attributes may be used to detect changes in compliance. However, in certain embodiments, the morphology of the plethysmographic signal may provide information about other physiological conditions. For example, these signal attributes may be used to provide information relevant to cardiovascular and central nervous toxicity associated with the intravascular injection of local anesthetic solutions. Also, these signal attributes may be used to provide information relevant to blood pressure and regional saturation to determine the depth of anesthesia. Further, these signal attributes may provide information about the character of the ejection from of the heart during systole. As mentioned above, information related to the baseline plethysmographic signal (e.g., specific signal attributes) and/or the baseline plethysmographic signal as well as subsequently obtained plethysmographic signals of the patient may be stored in the encoder 42 for access by the monitor 12 for processing.

Turning to the figures, FIG. 4A illustrates a plethysmographic signal 94 with multiple amplitudes. The plethysmographic signal 94 may include an ascending limb 96 and a descending limb 98. The ascending limb 96 may represent the systolic phase and the descending limb 98 the diastolic phase. Also, the plethysmographic signal 94 may include a dicrotic notch 100 usually present on the descending limb 98. The dicrotic notch 100 may be related to a sudden drop in pressure after systolic contraction caused by the back flow of blood into the arteries while the aortic valve is still closing. The amplitude of the plethysmographic signal 94, as represented by height 102 of the systolic peak 104, may correlate with perfusion. Increases in blood volume or stroke volume may increase the amplitude of the plethysmographic signal 94 which correlates to a more compliant tone or vasculature. Also, vasodilation due to anesthesia, for example, may increase the amplitude of the signal 94. Conversely the amplitude of the signal 94, as well as the compliance of the vasculature, may decrease along with decreases in blood volume and vasoconstriction. Reference numeral 106 represents the height of the dicrotic notch 100 and reference numeral 108 represents the height of diastolic peak 110. The ratio of the height 102 of the systolic peak 104 to the height 106 of the dicrotic notch 100 may be compared to measure the displacement of the dicrotic notch 100 in response to vasoactive drugs that alter vascular compliance or tone.

FIG. 4B illustrates a duration 112 of the plethysmographic signal 94 from the beginning 114 of the systolic phase to end 116 of the diastolic phase. When the vasculature is compliant and the blood volume is adequate, the duration 112 of the signal 94 may be longer. Conversely, with a less compliant vasculature and/or a lower blood volume, the duration 112 of the signal 94 may be shorter.

FIG. 4C illustrates an area 118 under the plethysmographic signal 94. Changes in stroke volume also may affect the area 118 under the waveform. The area 118, as illustrated, is defined as that area underneath the signal 94 from the beginning 114 of the systolic phase to the end 116 of the diastolic phase. Larger areas may be indicative of a more compliant vasculature and larger blood volume, while smaller areas may be indicative of a less compliant vasculature and/or smaller blood volume.

FIG. 4D illustrates changes in sharpness of the plethysmographic signal 94 from a broad waveform 120 to a narrower waveform 122. The broad, rounded waveform 120 represents an elastic (compliant) vasculature with adequate blood volume. The narrower, peaked waveform 122 represents a less compliant vasculature with less blood volume and narrower blood vessels. In addition, dicrotic notch 124 on the narrower waveform 122 is sharper than the dicrotic notch 126 on the broader waveform 120. The sharpening of the dicrotic notch 124 in the narrower waveform 122 may correlate to the constriction of blood vessels.

FIG. 4E illustrates changes in slope 128 and 130 of the ascending 96 and descending limbs 98 of the plethysmographic signal 94. As illustrated, the signal 94 includes a gradually sloped waveform 132 and a sharply-sloped waveform 134. The steepness of the slope 128 of the ascending limb 96 of the signal may correlate to the force of left ventricular contraction. A steep descending limb 98 of the signal 94 followed by a relatively prolonged baseline, as illustrated in the sharply-sloped waveform 134, may be related to inadequate blood volume relative to the compliance of the vasculature. The gradually, sloped waveform 132 with less steep limbs 96 and 98 may represent a more compliant vasculature with adequate blood volume.

FIG. 4F illustrates the movement of the dicrotic notch 100 along the plethysmographic signal 94. The signal 94 includes three different representative waveforms 136, 138, and 140. Typically the dicrotic notch 100 may be located on the descending limb 98 of the signal 94, towards or slightly above the middle as illustrated in waveform 138. However, during vasoconstriction (i.e., with a less compliant vasculature) the dicrotic notch 100 may be delayed and appear lower on the descending limb 98 as illustrated in waveform 140. Sometimes, during extreme vasodilation (i.e., with a more compliant vasculature), the dicrotic notch 100 may appear on the ascending limb 96 as illustrated in waveform 136.

FIG. 4G illustrates oscillation or twisting of the plethysmographic signal 94. The signal 94 includes three different representative waveforms 142, 144, and 146. Waveform 142 may represent the signal 94 free of any significant twisting. However, the breathing of the patient 40 may cause the signal 94 to oscillate or twist. For example, as illustrated in waveform 144, breathing may cause the slope 128 of the ascending limb 96 to decrease and the slope 130 of the descending limb 98 to decrease resulting in a clockwise twist in the signal 94. In addition, breathing may cause changes in slopes 128 and 130 to twist the signal 94 in a counter clockwise direction as illustrated with waveform 146. Inhalation and expiration during respiration may change the blood volume and, thus, vascular tone/compliance. For example, deep inhalation due to increased sympathetic activity may constrict arteries making them less compliant. However, during expiration, decreased sympathetic activity results in a decrease in vascular tone and an increase of blood flow into the arteries, and an increase in compliance.

As mentioned above, changes in the above described attributes of the plethysmographic signal may be correlated to a change in compliance. Various measurements of compliance as well as any changes in compliance may be provided on the display 20 of the monitor 14, so a clinician may quickly and easily understood any changes in compliance.

FIG. 5 illustrates an embodiment of a display 148 for compliance. The display 148 may display a current plethysmographic signal 150. In addition, the display 148 includes a graphical indicator 152 for various measurements of calculated compliance based on morphology of the plethysmographic signal 152 from the obtained baseline plethysmographic signal as described above. The graphical indicator 152 may include a measurement 154 for compliance measured as an increase in volume over a change in pressure (mL/mm Hg).

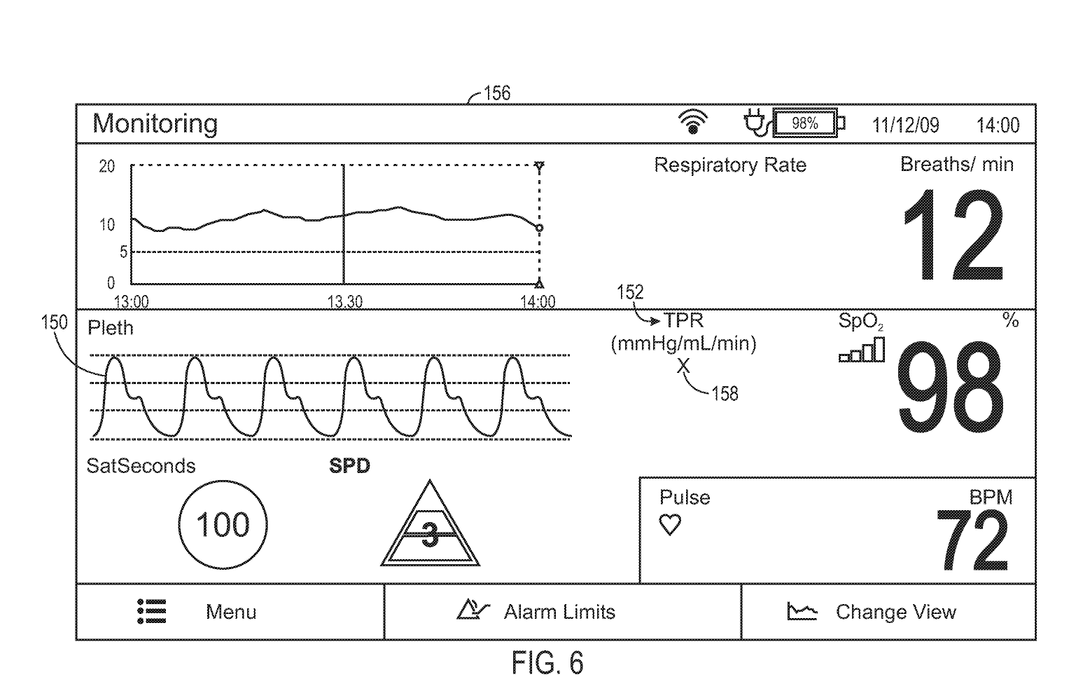

Alternatively, compliance may be measured as total peripheral resistance (TPR), FIG. 6 illustrates another embodiment of a display 156 for compliance. The graphical indicator 152 of display 156 may include a measurement for TPR 158. TPR is the sum of the resistance of all peripheral vasculature in the systemic circulation. The TPR may be measured as the change in pressure across the systemic circulation from a beginning point to an end point over the flow through the vasculature. The TPR may be an arbitrary value relative to the starting value for each individual. The measurement unit for TPR may be expressed in peripheral resistance units (mm Hg/mL/min). In certain embodiments, the display 156 may include pressure/volume curves or pressure/time curves for compliance.

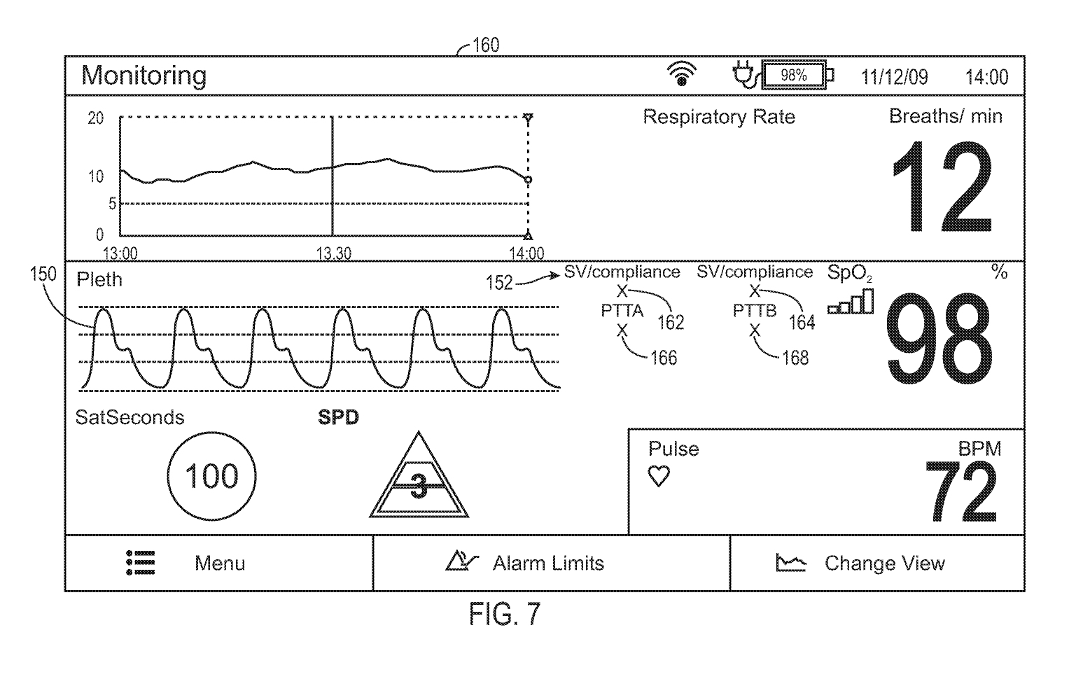

When plethysmographic signals are obtained from multiple locations, further information may be displayed with respect to compliance of a particular arterial tree. FIG. 7 illustrates a further embodiment of a display 160 for compliance. The comparison of particular arterial trees may be shown on the display 160 if using sensors placed in different areas of the body (e.g., the finger and the forehead). The ratio of stroke volume output to compliance of a particular arterial tree may be measured and compared to another arterial tree to assess differences and similarities. Graphical indicator 152 may include a ratio 162 of stroke volume output over compliance for a central arterial tree and a similar ratio 164 for a peripheral arterial tree. In certain embodiments, the graphical indicator 152 may also include measurements 166 and 168 for pulse transit time for both the central and more peripheral arterial tree, respectively. In terms of pulse transit time, a slower transmission of a pulse wave may indicate greater compliance within an arterial tree. In addition, when using multiple sensors, if one sensor has a slower transit time relative to itself and another sensor, then the compliance change occurred in only one of the arterial trees.

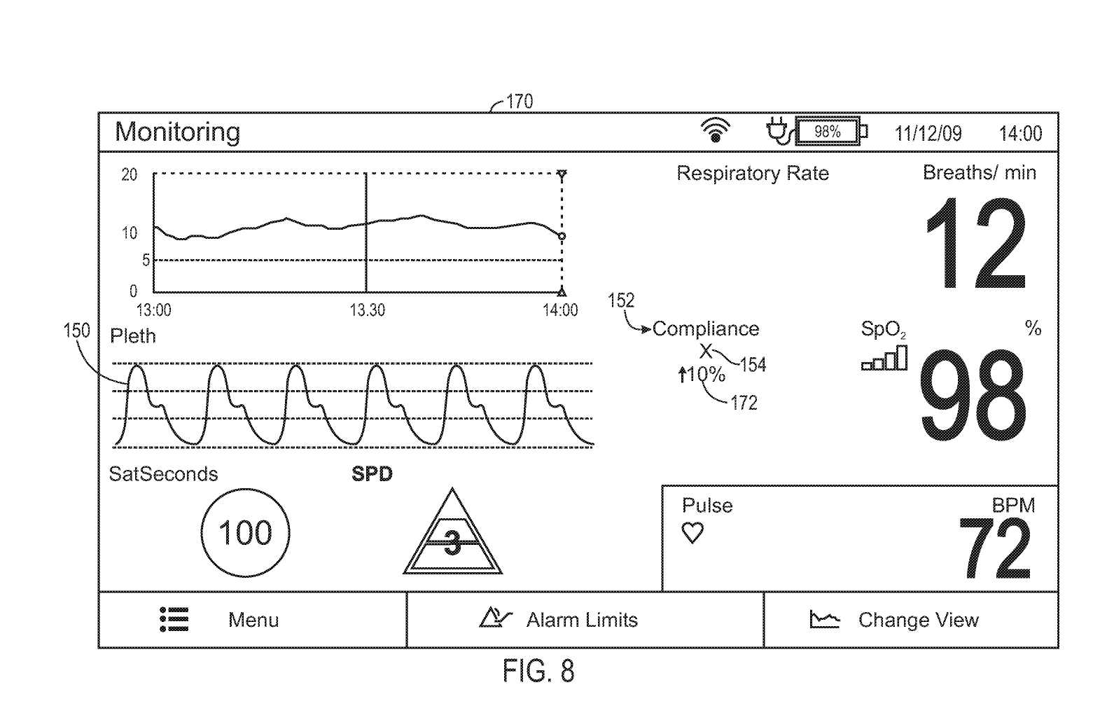

As mentioned above, the monitor 14 may also provide alarms for changes in compliance. FIG. 8 illustrates an embodiment of a display 170 for changes in compliance. As illustrated the display 170 includes graphical indicator 152 as shown in FIG. 5. In addition, the graphical indicator 152 may include a measurement 172 for compliance. Changes in compliance may be displayed as a percentage. In addition, the measurement 172 may include an up arrow to indicate increases in compliance and a down arrow to indicate a decrease in compliance.

A clinician may be more concerned with rapid changes in compliance. Thus, alarm limits may be incorporated to reflect this concern. Thus, certain percent changes in compliance over a given period of time may trigger different levels of alarms. In certain embodiments, significant percent changes regardless of time may trigger an alarm. For example, a 50% increase in compliance in 10 seconds or less may trigger a high level alarm, a 50% increase in compliance in 10 seconds or more may trigger a low level alarm, and a 75% increase in compliance may trigger a high level alarm. As for a decrease in compliance, a 30% decrease in compliance may raise a flag, a 50% decrease in compliance may trigger a low level alarm, and a 75% decrease in compliance in 10 seconds or less may trigger a high level alarm. To visually indicate the alarm, the measurement 172 may be color coded. For example, green, yellow, orange, and red may represent normal compliance, a flag, a low level alarm, and a high level alarm, respectively. In other embodiments, an audible alarm via speakers 22 may be provided separately or in conjunction with the visual alarm.

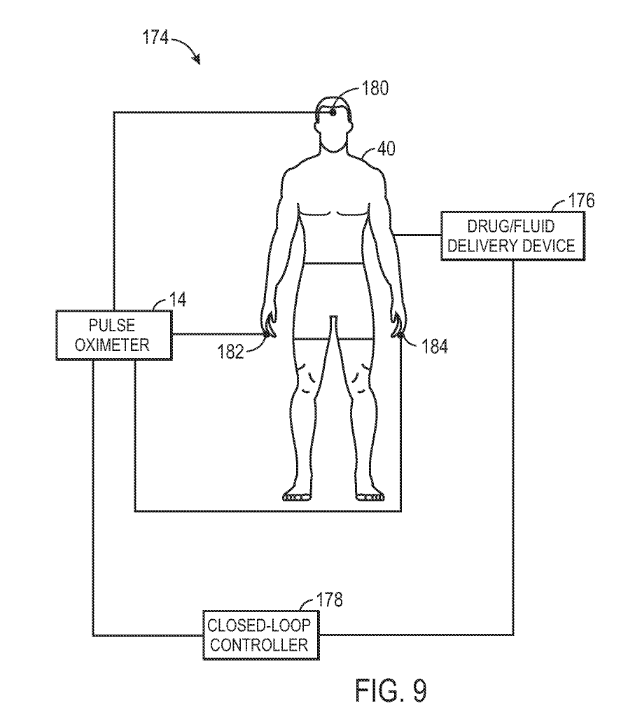

As an alternative to providing alarms, the degree of compliance may be regulated via the administration of a vasoactive stimulus. FIG. 9 illustrates the use of a closed-loop system 174 to manage compliance. The closed-loop system 174 may include a drug/fluid delivery device 176 to administer (e.g., intravenously) a controlled amount of substance, such as a vasoactive stimulus to the patient 40. For example, the vasoactive stimulus may include a vasodilator or vasoconstrictor. Also, the vasoactive stimulus may include an anesthetic solution. The drug/fluid delivery device 176 may include an input for the user to enter the amount of substance to be administered to the patient 40. The amount of substance delivered may be altered by the device 176 in response to signals received from the pulse oximeter 14 and/or a closed-loop controller 178. The pulse oximeter 14 may calculate compliance, among other physiological parameters, via signals received from sensors 180, 182, and 184 coupled to the patient 40. Sensors 180, 182, 184 may allow compliance measurements for different arterial trees. As illustrated, three sensors 180, 182, and 184 are included, but in alternative embodiments only a single sensor may be used or any other number of sensors. The pulse oximeter 14 may use the acquired signals, as described above, to determine compliance.

The closed-loop controller 178 may be coupled to the pulse oximeter 14. The closed-loop controller 178 may include a set compliance point or set compliance range for the patient 40. The set compliance point or range may be provided to the closed-loop controller 178 by the pulse oximeter 14. Also, the pulse oximeter 14 may provide to the closed-loop controller 178 the current compliance level of the patient 40. In response to receiving the current compliance level, the closed-loop controller 178 may send a signal to the drug/fluid delivery device 176 to administer a specific substance to the patient 40 in order to bring or to maintain the patient's compliance at the set compliance point or within the set compliance range. For example, the patient 40 may have a current compliance level of 6.0 mL/mm Hg. Upper and lower limits of the set compliance range may be set at 10.0 mL/mm Hg and 4.0 mL/mm Hg, respectively. The closed-loop controller 178 may send a signal to the drug/fluid delivery device 176 to administer the substance to the patient 40 to bring the compliance level within the lower limit of 4.0 mL/mm Hg of the set compliance range. Alternatively, the current compliance level of the patient 40 may be at 5.0 mL/mm Hg but trending downward. In this scenario, the closed-loop controller 178 may send a signal to the drug/delivery device to administer a substance to the patient 40 to maintain the compliance level with the set range limit. The above values are intended only to serve as examples. In other embodiments, the set compliance point or set compliance range may vary.

The closed-loop controller 178 may include a memory storing an algorithm configured to calculate adjustments for inducing, maintaining, and/or controlling physiological parameters of the patient 40. Such algorithms (e.g., P, PD, PI, and PID algorithms) may be utilized to bring the patient's physiological parameters to a desired state. For example, predefined proportional, integral, and/or derivative factors may be designated to facilitate tuning control loops based on physical characteristics of the patient 40 (e.g., age or weight). In a specific example, certain integral factors for designated patient types may be used in a PI controller algorithm to make sure a certain patient compliance level is approached steadily. Additionally, other loop tuning features (e.g., a derivative factor) may be utilized to improve control.

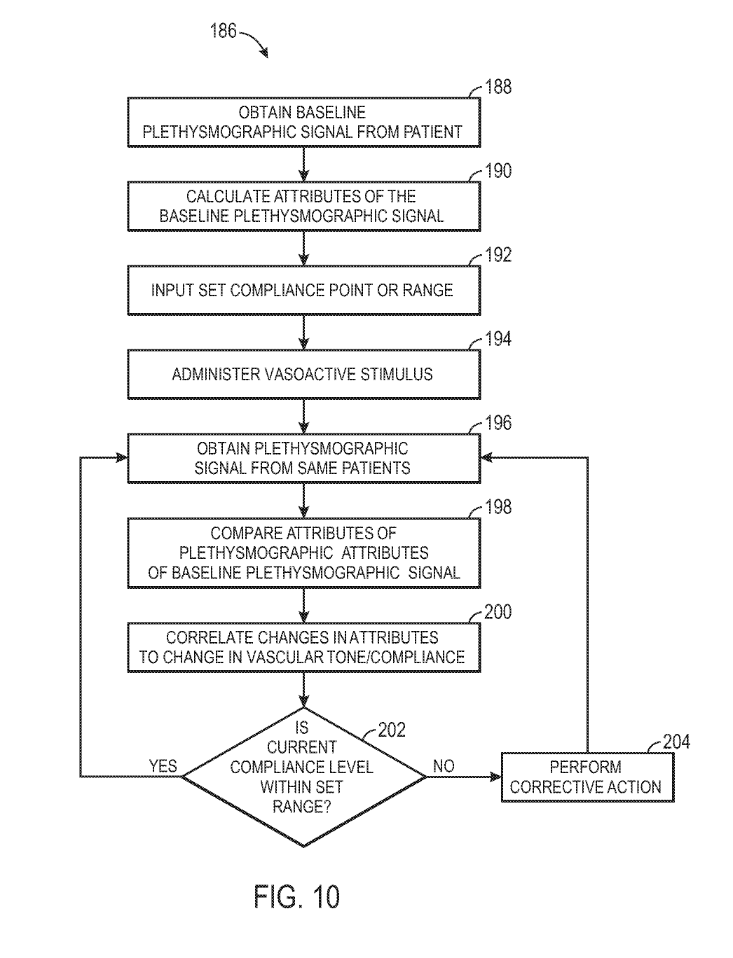

As discussed above, a vasoactive stimulus may be administered to control the compliance level of the patient 40. FIG. 10 illustrates a method 186 for determining and managing compliance using embodiments described above and the closed-loop system 174 embodied in FIG. 9. The method 186 may begin similar to method 78 above with obtaining a baseline plethysmographic signal from the patient 40 (block 188). The baseline plethysmographic signal may be the original signal or a higher resolution signal. Upon obtaining the baseline signal, attributes of the baseline plethysmographic signal may be calculated (block 190), as described above. Prior to administering a vasoactive stimulus, a desired compliance level or range may be input (block 192) into the pulse oximeter 14. The user may enter the desired set compliance point or range. Alternatively, an algorithm may be used to take patient specific parameters (e.g., age and weight) to calculate the appropriate compliance point or range to be used by the pulse oximeter 14 and/or closed-loop controller 178. A set compliance range may also be determined from the set compliance point. Subsequent to setting the desired compliance level or range and calculating the attributes of the baseline signal, a vasoactive stimulus or drug, as described above, may be applied to the patient 40 (block 194).

Following application of the vasoactive stimulus, a plethysmographic signal may be obtained from the same patient 40 (block 196). As with the baseline plethysmographic signal, the plethysmographic signal may be the original signal or a higher resolution signal. Also, similarly, the same attributes may be calculated from the plethysmographic signal as were calculated from the baseline plethysmographic signal. Following processing, the calculated attributes from the plethysmographic signal may then be compared to calculated attributes of the baseline signal (block 198), as described above. Upon comparing the attributes from both signals, changes in these attributes may be correlated to a change in vascular tone/compliance (block 200), as described above.

After determining the change in compliance, the pulse oximeter 14 may determine whether the current compliance level is outside the set compliance range (block 202). If the current compliance level does not fall outside the set compliance range, then the pulse oximeter 14 may continue to obtain the plethysmographic signal (block 196) to monitor the compliance level. If the current compliance level does fall outside the set compliance range, corrective action may be performed (block 204) and the plethysmographic signal obtained again (block 196). Corrective action may include administering a vasoactive stimulus to increase or decrease the compliance to the desired compliance level. The corrective action may be under the control of closed-loop controller 178 and administered via drug/fluid delivery device 176, as described above. The corrective action may be used to return the compliance level of the patient 40 within the set compliance range without the need of a caregiver's presence. In another embodiment, the closed-loop corrective action may be used to maintain the compliance level of the patient 40 within the set compliance range.

The above embodiments describe analyzing attributes of the original plethysmographic signal or a related higher resolution signal for determining and indicating changes to vascular tone or compliance in response to a vasoactive stimulus. In other embodiments, the signal attributes may be used to determine and indicate changes to cardiovascular and central nervous system toxicity associated with the intravascular injection of local anesthetic solutions. In further embodiments, the signal attributes may be used to determine and indicate changes to blood pressure and regional saturation to determine the depth of anesthesia. It should be noted that, in order to measure blood pressure, embodiments of the present disclosure may utilize systems and methods such as those disclosed in U.S. Pat. No. 7,455,643 and U.S. Pat. No. 6,599,251, and each are incorporated herein by reference in their entirety for all purposes.

What is claimed is:

1. A monitor comprising:
a processing unit configured to:
calculate one or more baseline attributes of a baseline plethysmographic signal obtained from a patient;
compare one or more calculated attributes of a plethysmographic signal to the one or more calculated baseline attributes; and
correlate changes in the one or more calculated attributes from the one or more calculated baseline attributes to a change in vascular compliance; and
a display unit configured to provide an indication of the change in vascular compliance.

2. The monitor of claim 1, wherein the baseline plethysmographic signal or the baseline attributes are obtained from a memory of a sensor.

3. The monitor of claim 1, wherein the baseline plethysmographic signal and the plethysmographic signal comprise continuous wavelet transformed signals.

4. The monitor of claim 1, wherein the attributes of the baseline plethysmographic signal and the plethysmographic signal comprise one or more amplitudes of one or more peaks, a length in duration from a beginning of a systolic phase to an end of a diastolic phase, an area under a curve, a position of a dicrotic notch, slopes of ascending and descending limbs, or movement of the dicrotic notch.

5. The monitor of claim 1, wherein the processing unit is configured to trigger an alarm when the change in vascular compliance exceeds a certain threshold.

6. The monitor of claim 5, wherein the threshold comprises a percent change in the vascular compliance.

7. The monitor of claim 5, wherein the display unit is configured to present the alarm in response to the trigger from the processing unit.

8. The monitor of claim 1, wherein the plethysmographic signal is obtained from the patient after the administration of a vasoactive stimulus.

9. A system comprising:
a sensor capable of generating a physiological signal;
a processing unit configured to:
calculate one or more baseline attributes of a baseline physiological signal obtained from a patient;
compare one or more calculated attributes of a subsequent physiological signal obtained from the patient to the one or more calculated baseline attributes; and
correlate changes in the one or more calculated attributes from the one or more calculated baseline attributes to a change in vascular compliance.

10. The system of claim 9, wherein the baseline physiological signal and the subsequent physiological signal comprise continuous wavelet transformed signals.

11. The system of claim 9, wherein the attributes of the baseline physiological signal and the subsequent physiological signal comprise one or more amplitudes of one or more peaks, a length in duration from a beginning of a systolic phase to an end of a diastolic phase, an area under a curve, a position of a dicrotic notch, slopes of ascending and descending limbs, or movement of the dicrotic notch.

12. The system of claim 9, wherein the processing unit is configured to obtain the baseline physiological signal or the baseline attributes from a memory of the sensor.

13. The system of claim 9, comprising a display unit configured to provide an indication of the change in vascular compliance.

14. The system of claim 9, wherein the processing unit is configured to trigger an alarm when the change in vascular compliance exceeds a certain threshold.

15. The system of claim 14, comprising a display unit configured to present the alarm in response to the trigger from the processing unit.

16. The system of claim 14, wherein the threshold comprises a percent change in the vascular compliance.

17. The system of claim 9, wherein the subsequent physiological signal is obtained from the patient after the administration of a vasoactive stimulus.

18. A method, comprising:
obtaining a baseline plethysmographic signal from a patient;
calculating one or more attributes of the baseline plethysmographic signal;
administering a vasoactive stimulus to the patient;
obtaining a subsequent plethysmographic signal from the patient;
calculating one or more attributes of the subsequent plethysmographic signal;
comparing the one or more calculated attributes of the subsequent plethysmographic signal to the one or more calculated attributes of the baseline plethysmographic signal; and
correlating changes in the one or more calculated attributes to a change in vascular compliance in the patient.

19. The method of claim 18, comprising providing an indication of change in the vascular compliance on a display.

20. The method of claim 18, comprising triggering an alarm when the change in vascular compliance exceeds a certain threshold.
